(19) United States
(12) Patent Application Publication
(10) Pub. No.: US 2017/0007465 A1
(43) Pub. Date: Jan. 12, 2017

(54) Sock And Method For Creating A Substantial Squeezing Pressure On One Or More Individual Toes Of A Wearer

(71) Applicant: Kevin Edwards, Cookeville, TN (US)

(72) Inventor: Kevin Edwards, Cookeville, TN (US)

(21) Appl. No.: 15/203,903

(22) Filed: Jul. 7, 2016

Related U.S. Application Data
(60) Provisional application No. 62/190,354, filed on Jul. 9, 2015.

(51) Int. Cl. A61F 13/06; A61F 5/01; A61F 5/058; A61F 13/08

(52) U.S. Cl. CPC A61F 13/068; A61F 13/08; A61F 5/019; A61F 5/0585; A61F 5/0195

(57) ABSTRACT

A sock has individual toe pockets attached to a foot portion of the sock receiving the toes of a wearer which are elastically stretchable to exert a substantial squeezing pressure on an inserted toe sufficient to prevent swelling of a toe as after surgery thereon or to improve blood circulation to the toes. Optionally, splint pockets are attached on the outside of one or more of the toe pockets.

### CROSS REFERENCE TO RELATED APPLICATIONS

This application claims the benefit of U.S. provisional application No. 61/190,354 filed on Jul. 9, 2015.

### BACKGROUND OF THE INVENTION

This application concerns applying a substantial squeezing compression of any of a person's toes so as to prevent swelling, as is typically needed after surgery on a toe.

The usual practice is to wrap the toe or toes with gauze. This requires multiple turns and is sufficiently bulky to cause some uncomfortable spreading of the toes.

This bulkiness also may prevent wearing of a shoe on the affected foot.

Furthermore, such a wrapping is difficult to keep in place, particularly on the little toe.

There are various other reasons for applying a substantial squeezing pressure on a person's toes, i.e., to improve blood circulation to the toes, for diabetics, and those suffering from peripheral vascular disease; treatment of hammer toe or turf toe; to stabilize a straightened broken toe, and after surgeries for bunion and bone spur removal.

It is an object of the present invention to provide a sock and method which exerts sufficient squeezing compression to any individual toe or to two or more toes of a wearer to prevent or limit swelling of the toe or toes of a wearer and which allows the wearing of a shoe on an affected foot, and is securely retained in position on the affected toe or toes.

### SUMMARY OF THE INVENTION

The above object and other objects which will be understood by those skilled in the art are achieved by a foot wearable article or sock having five individual toe pockets configured to fit the respective toes or a foot of a wearer. The toe pockets are made of a flexible and elastic material capable of exerting a substantial compressive squeezing pressure on a toe inserted in one of the toe pockets where the toe pockets are smaller than the toes such that stretching of the toe pocket material occurs to a degree which creates the proper level of squeezing force on the toe to prevent or substantially lessen the degree of swelling after surgery.

The sock has a foot portion to which the several toe pockets are attached in the proper positions to receive the respective toe designed to be fit thereto.

Each of the toe pockets may be connected to an end of the foot portion by a seam extending around to encircle the connected inner end of the toe pockets so that individual toe pockets not needed by the wearer can be cut off as with scissors while leaving the remaining pockets intact preventing unraveling of the woven material away from a cut edge.

The seams are not needed for socks contemplated as being worn by a diabetic or others suffering from which condition would not involve removal of any toe pockets.

The material used for the sock is an elastic woven material but preferably made from, Spandex (also known as lycra or elastone) and also sold under various other trade names in foreign countries, or silk fabric. A nonwoven flexible elastic sheet material such as rubber may be used but is not preferred. The elastic woven Spandex material is preferred due to its strength and elasticity which is able to exert a substantial squeezing compressive force or the toes of a wearer.

The toe pockets are not as bulky as gauze wrappings while being capable of generating sufficient compression or squeezing pressure on the toes to alleviate the tendency for selling to occur.

The toe pockets may be sized to apply a maximum compression at the outer end of the toe with the squeezing pressure decreasing along the length of the toe in a direction towards the foot to aid in improving blood circulation to and from the toes of a wearer.

The toe pockets may also each have one or a pair of an open ended splint pockets extending along the length of the outside of each toe pocket, each allowing insertion of a straightening splint to hold a broken toe in the associated pocket in a straightened set condition.

### DESCRIPTION OF THE DRAWINGS

FIG. 1 is a pictorial view of a toe sock according to the invention being worn on the foot of a user.

FIG. 2 is an enlarged top view of a portion of the sock shown in FIG. 1 with an added splint pocket.

FIG. 2A is a bottom view of the portion of the sock shown in FIG. 2.

FIG. 3 is an enlarged fragmentary view of the sock shown in FIG. 1 with two toe pockets removed.

FIG. 4 is an enlarged fragmentary view of a modified form of the sock according to the invention with a splint pocket associated with the toe pockets partially broken away to show a splint inserted therein.

FIG. 5 is a bottom view of the sock portion shown in FIG. 4.

FIG. 6 is a top view of the sock portion shown in FIG. 5.

FIG. 7 is a fragmentary pictorial view of a sock portion according to an alternate embodiment of the invention.

### DETAIL DESCRIPTION

In the following detailed description, certain specific terminology will be employed for the sake of clarity and a particular embodiment described in accordance with the requirements of 35 USC 112, but it is to be understood that the same is not intended to be limiting and should not be so construed inasmuch as the invention is capable of taking many forms and variations within the scope of the appended claims.

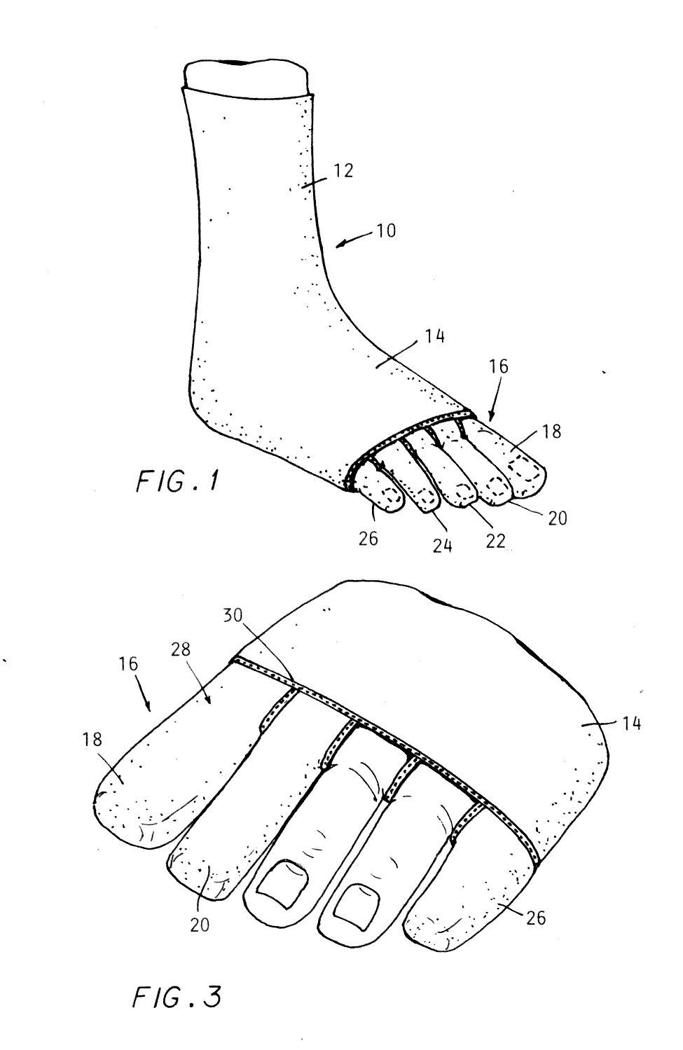

Referring to the drawings and particularly FIGS. 1 and 3, a sock 10 according to the invention is shown being worn on the foot of a wearer. The sock 10 has a conventional upper portion 12 receiving the ankle and the lower part of the calf of the wearer. A foot portion 14 encloses the foot of the wearer.

The present invention is concerned with a toe portion 16 forming a part of the foot portion, which is comprised of five compression toe pockets 18, 20, 22, 24, 26 integral with an outer portion 28 of the foot portion 14 which partially encloses the portion of the foot from which the wearer's toes project. The outer end of the foot portion 28 of the toe portion 16 is attached by an upper seam segment 30 to the conventional upper part of the foot portion 14.

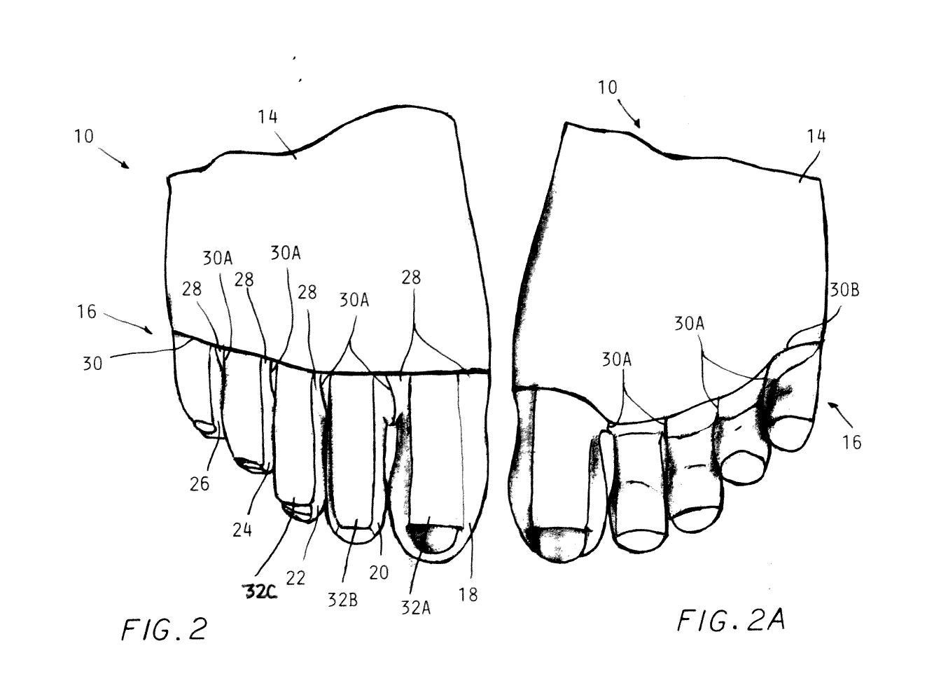

As best seen in FIGS. 2 and 2A, there are additional seam segments 30A which extend from seam 30 and extend towards and between each of the respective toe pockets 18-26.

The segments 30A each extend to a bottom seam 30B extending across the bottom of foot portion 28.

Thus each pocket 18-26 is completely encircled by the seam segments 30, 30A, 30B for a purpose described below.

Each toe pocket 18-26 is configured to be tightly fit to a respective toe of the wearer, the tightness of fit and elasticity of the material from which the toe pockets 18-26 are made enabling a substantial squeezing compression to be exerted on any or all of the inserted toes so as to at least substantially counter the tendency for swelling to occur after surgery on a toe.

This squeezing pressure also may promote blood circulation to and from the toes as may be beneficial to diabetics.

This substantial squeezing pressure on the toes requires a strong elastic material such as Spandex, also known as lycra and generally referred to as elastone which is able to exert a substantial squeezing compression on toes inserted in the pockets 18-26.

The degree of squeezing compression should be set to be at a level that is medically indicated for the intended purpose. The material thickness and the degree of expansion produced by the size of the pocket when the toes are inserted should be selected so as to affect the medically indicated degree of compression. This substantially exceeds the mere snugness of a stretching of the conventional material of a sock when put on.

The seam segments 30, 30A, 30B allow one or more of the toe pockets 18-26 to be cut away if not needed without causing unraveling of the material which make up the remaining toe pockets 18-26 or the foot portion 14 so as to minimize the bulk of the sock in the situation in which all of the toes do not need to be squeezed.

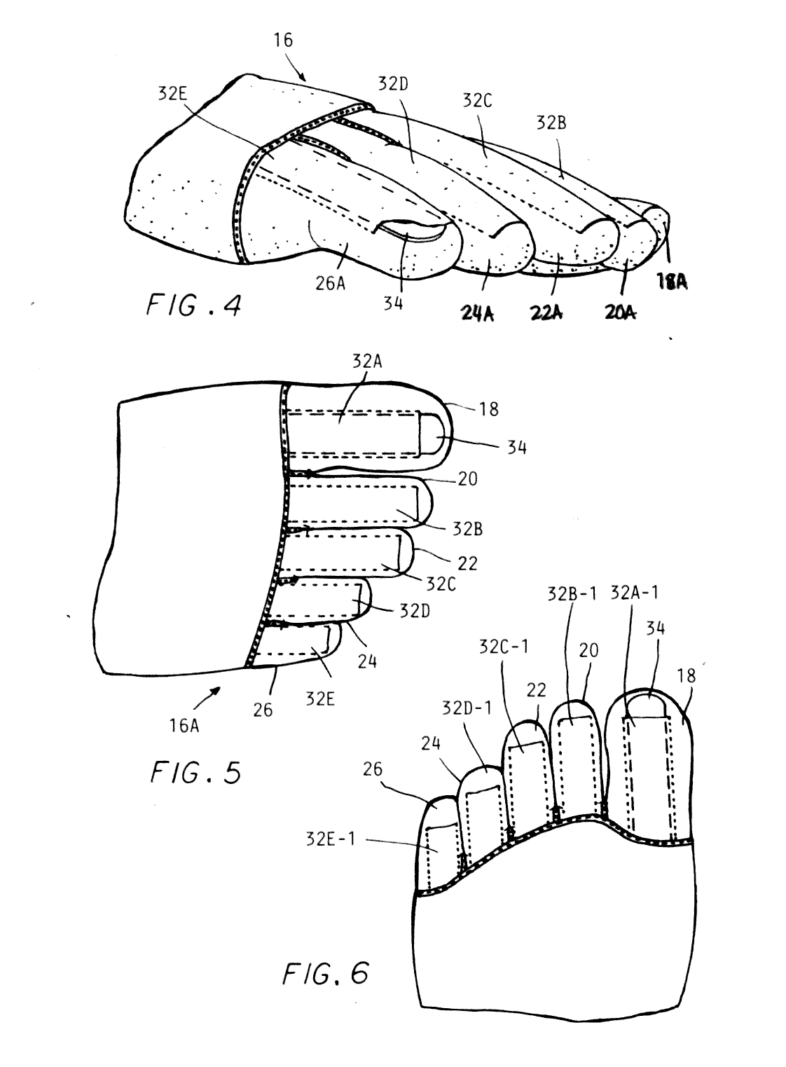

As best seen in FIGS. 4, 5 and 6 the sock 10 can also be fitted with splint pockets 32A-32E on the top of each pocket 18-26 and/or pockets 32A-1 to 32E-1 on the bottom of each toe pocket 18-26 allowing stiff splints 34 to be inserted to hold a broken toe in a set position.

The toe compression could be reduced to be just sufficient to securely hold the position of an inserted splint 34 if only the splints 34 are needed.

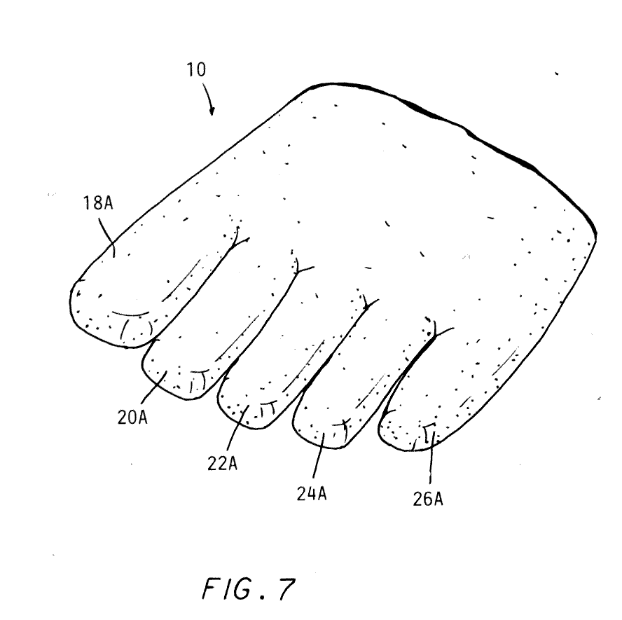

FIG. 7 shows a seamless version of a sock 10A which may be use by diabetics in which all of the toe pockets 18A-26A are integral with the remaining portions of the sock 10A. Removal of individual toe pockets 18A-26A would not be anticipated for such use and thus a seam would not be necessary.

1. A foot wearable sock for producing compression of one or more of a wearer's toes including a portion able to receive and be fitted to an enclose a wearer's foot, a toe section extending from said foot portion disposed comprised of a series of toe pockets each able to receive and be tightly fit to a respective on a wearer's toe, said toe pockets each configured to exert a sufficient force on said inserted toe to able to exert a squeezing compression of said toe to a sufficient degree to substantially counter any tendency of swelling of as for a toe which has undergone surgery.

2. The sock according to claim 1 wherein said article includes an upper portion extending up from said foot portion adapted to receive a lower leg of a wearer.

3. The sock according to claim 1 wherein each of said toe pockets is attached to said foot portion by a respective seam encircling each of said toe pockets at a location adjacent said foot portion whereby one or more of said toe pockets can be cut off without making adjacent foot portions vulnerable to unraveling.

4. The sock according to claim 1 wherein said sock toe pockets are comprised of an elastic woven material able to exert a substantial squeezing pressure on an inserted toe of a wearer.

5. The sock according to claim 4 wherein said elastic woven material comprises Spandex.

6. The sock according to claim 1 wherein one or more of said toe pockets has an attached splint pocket attached to the outside of said toe pocket extending along the length thereof.

7. The sock according to claim 6 wherein one or more of said toe pockets has an additional splint pocket attached to the outside of a toe pocket.

8. The sock according to claim 1 wherein one or more of said toe pockets are woven so as to apply a varying squeezing pressure on an inserted toe which is at a maximum at the end of the toe and decreases along the length thereof.
